(19) United States
(12) Patent Application Publication
(10) Pub. No.: US 2022/0339417 A1
(43) Pub. Date: Oct. 27, 2022

(54) IMPLANTABLE SUBSTANCE DELIVERY VEHICLE AND MANUFACTURING METHOD THEREFOR

(71) Applicant: SNVIA CO., LTD., Busan (KR)

(72) Inventors: Seung Yun YANG, Daegu (KR); Sang Gu YIM, Busan (KR); Eun Nam TAK, Gyeongsangnam-do (KR)

(73) Assignee: SNVIA CO., LTD., Busan (KR)

(21) Appl. No.: 17/754,409

(22) PCT Filed: Sep. 2, 2020
(86) PCT No.: PCT/KR2020/011746

§ 371 Date: Mar. 31, 2022

(30) Foreign Application Priority Data
Oct. 11, 2019 (KR) 10-2019-0126018

(51) Int. Cl. A61M 37/00

(52) U.S. Cl. CPC A61M 37/0015; A61M 2037/0053; A61M 2037/0023

(57) ABSTRACT

An implantable substance delivery vehicle of the present invention comprises: a pin comprising a bevel tip and an engraved groove on the surface of the bevel tip; and a substance to be delivered into the body, inserted so as so as to protrude outwardly from the tip, wherein, when the implantable substance delivery vehicle is used for implantation, the substance to be delivered into the body, inserted so as so as to protrude, is caught in tissue so as to be readily detached.

### FIELD OF THE INVENTION

The present disclosure relates to an implantable substance delivering carrier, and a method for manufacturing the same. More specifically, the present disclosure relates to an implantable substance delivering carrier in which an engraved groove is defined in a surface of a bevel tip such that a delivery target substance may be stably delivered to an accurate location.

### DESCRIPTION OF RELATED ART

A scheme to effectively deliver a target substance (e.g., a drug, etc.) into an animal's body may be broadly classified into a delivery system using a carrier such as a polymer and a delivery device system using a fusion technology. The delivery system using the polymer may be classified into a scheme in which a drug binds to a polymer, and a scheme using a formulation in which a drug is supported on nano- or micro-sized polymer particles. On the other hand, the delivery device system includes an implantable implant requiring surgical intervention or various patches that promote drug absorption. Recently, a patch that delivers a target substance through a tissue (skin or mucous membrane) using a micro-needle that may minimize pain and tissue damage has been on the spotlight.

The microneedle has a microneedle shape having a length and a thickness of several hundred micrometers. In general, a scheme of using the microneedle includes putting a target substance in a polymer solution constituting the microneedle, and then filling a desired mold with the solution, and drying the mold such that a target substance is put into the microneedle that dissolves in a body, or coating the target substance on an insoluble microneedle using a polymer substance and drying the same and inserting the microneedle into the skin tissue such that the target substance is delivered into the skin via a process of dissolving the same in the body. However, the preparation method accompanied by the drying process may induce the denaturation of a biological substance that is very sensitive to a surrounding environment, such as exosomes, proteins, or cells. Further, when the polymer is not dissolved quickly, a large amount of the target substance may not be delivered to a large area in a quantitative manner. Further, there is a problem that it is not easy to supply the target substance to a desired location. Thus, research and development of a new type of a needle to solve this problem are required.

### SUMMARY OF THE INVENTION

One purpose of the present disclosure is to provide an implantable drug delivery technology and an implantable substance delivery device in which various drugs including a bio-substance are desorbed from a solid (including hydrogel type) drug delivery carrier carrying thereon the various drugs including a bio-substance therefrom and then the drugs are implanted into a tissue.

Another purpose of the present disclosure is to provide a method for manufacturing an implantable substance delivering carrier.

One aspect of the present disclosure provides an implantable substance delivering carrier comprising: a pin including a bevel tip and a groove engraved in a surface of the bevel tip; and a delivery target substance inserted into the engraved groove so as to protrude outwardly from the tip.

In one implementation of the implantable substance delivering carrier, the delivery target substance has a spherical or oval shape.

In one implementation of the implantable substance delivering carrier, the delivery target substance includes exosome, protein, peptide, spheroid cell body, hydrogel or dried solid body.

In one implementation of the implantable substance delivering carrier, the delivery target substance has a form of a core-shell in which the delivery target substance is located in the core and a biodegradable polymer or hydrogel surrounding the core is located in the shell.

In one implementation of the implantable substance delivering carrier, the implantable substance delivering carrier further includes a coating layer covering the delivery target substance and an entirety or a portion of the surface of the bevel tip.

In one implementation of the implantable substance delivering carrier, the delivery target substance is inserted into the grove so as to protrude outwardly beyond an imaginary line, wherein the line extends upwardly from a shorter side among a pair of parallel sides of the pin extending in a vertical direction in a vertical cross section of the pin.

In one implementation of the implantable substance delivering carrier, the engraved groove has an L-shape or an L-shape with a rounded corner in a vertical cross-section of the pin.

In one implementation of the implantable substance delivering carrier, the engraved groove is formed in the surface of the tip closer to a shorter side among a pair of parallel sides of the pin extending in a vertical direction in a vertical cross section of the pin.

In one implementation of the implantable substance delivering carrier, the surface face of the bevel tip is inclined at an angle of 45° to 80°.

In one implementation of the implantable substance delivering carrier, a length of the pin is in a range of 300 μm to 30 cm.

In one implementation of the implantable substance delivering carrier, the length of the pin is in a range of 700 μm to 5 mm.

In one implementation of the implantable substance delivering carrier, a thickness of the pin is in a range of 200 μm to 3.4 mm.

In one implementation of the implantable substance delivering carrier, a diameter of the engraved groove is in a range of 50 μm to 1 mm.

In one implementation of the implantable substance delivering carrier, a diameter of the engraved groove is in a range of 100 μm to 500 μm.

In one implementation of the implantable substance delivering carrier, a depth of the engraved groove is in a range of 50 μm to 2 mm.

In one implementation of the implantable substance delivering carrier, a depth of the engraved groove is in a range of 100 μm to 700 μm.

Another aspect of the present disclosure provides a method for manufacturing the implantable substance delivering carrier as described above, wherein the method comprises: dripping the delivery target substance dispersed in a polymer solution into the engraved groove of the pin; and drying the solution.

In one implementation of the method, the method further comprises forming a coating layer covering the delivery target substance and an entirety or a portion of the surface of the bevel tip.

In one implementation of the method, the polymer solution includes hyaluronic acid methacrylate.

According to the implantable substance delivering carrier according to the present disclosure, and the method for manufacturing the same, the substance to be delivered to the body, that is, the delivery target substance may be precisely implanted to a desired location. The delivery target substance may not be subjected to stress during the implantation, and thus may not be damaged and thus may be implanted stably. In addition, a large amount of the delivery target substance may be implanted at once, and may be implanted quantitatively and to a large area.

### BRIEF DESCRIPTION OF THE DRAWINGS

FIG. 1(a) to (b) are views showing an embodiment of an implantable substance delivering carrier according to the present disclosure, and a method for manufacturing the same.

FIG. 2(a) to (d) are diagrams for illustrating an engraved groove according to the present disclosure, and show a vertical cross-section of a pin according to the present disclosure.

FIG. 3 is a diagram for illustrating a process of implanting a delivery target substance using an implantable substance delivering carrier according to the present disclosure.

FIG. 4(a) to (b) are diagrams for illustrating a process of implanting a lyophilized product of a coated drug.

FIG. 5 is a diagram showing an implantable substance delivering carrier for implantation of a large amount of delivery target substance according to the present disclosure.

FIG. 6 is a diagram for illustrating an implantable substance delivering carrier prepared according to Example 1 of the present disclosure.

FIG. 7(a) to (b) are diagrams showing a result of evaluating a cell viability regarding an implantable substance delivering carrier according to the present disclosure.

FIG. 8 is a diagram showing a result of evaluating implantation efficiency of an implantable substance delivering carrier according to the present disclosure.

### DETAILED DESCRIPTION OF THE INVENTION

Hereinafter, an embodiment of the present disclosure will be described in detail with reference to the accompanying drawings. The present disclosure may be variously modified and may take many forms. Thus, specific embodiments will be illustrated in the drawings and described in detail herein. However, the specific embodiments are not intended to limit the present disclosure thereto. It should be understood that all changes, equivalents thereto, or substitutes therewith are included in a scope and spirit of the present disclosure. In describing the drawing, similar reference numerals are used for similar components.

The terminology used herein is for the purpose of describing particular embodiments only and is not intended to be limiting of the present disclosure. As used herein, the singular forms “a” and “an” are intended to include the plural forms as well, unless the context clearly indicates otherwise. It will be further understood that the terms “comprises”, “comprising”, “includes”, and “including” when used in this specification, specify the presence of the stated features, integers, operations, elements, and/or components, but do not preclude the presence or addition of one or greater other features, integers, operations, elements, components, and/or portions thereof.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

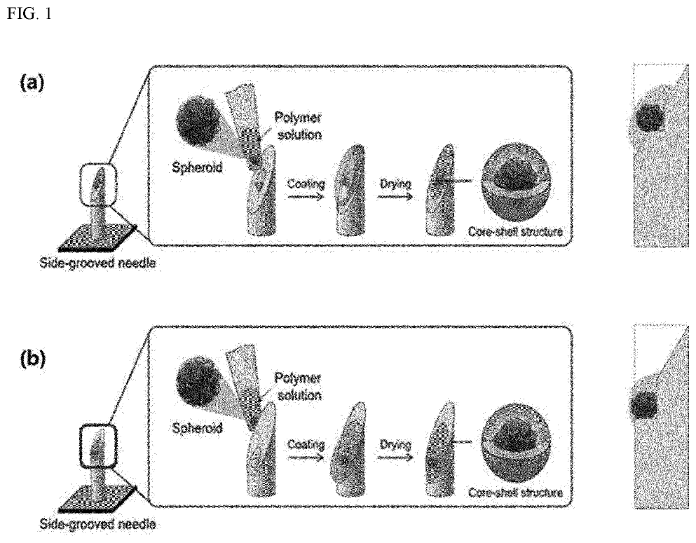

FIG. 1 is a view showing an embodiment of an implantable substance delivering carrier according to the present disclosure, and a method for manufacturing the same. Hereinafter, with reference to FIG. 1, the implantable substance delivering carrier according to the present disclosure, and the method for manufacturing the same will be described.

Referring to FIG. 1, the implantable substance delivering carrier according to the present disclosure includes a pin including a bevel tip and a groove engraved in a surface of the bevel tip; and a delivery target substance inserted into the engraved groove so as to protrude outwardly from the tip.

The pin may have a blade surface formed at a distal end thereof, and may have a length of 300 μm to 30 cm, and preferably, 700 μm to 5 mm. Further, the pin may have a thickness of 200 μm to 3.4 mm.

The surface of the bevel tip may mean a blade surface formed at the distal end of the pin. The surface of the bevel tip may have an inclination of 45° to 80°.

The engraved groove is a groove formed in the surface of the bevel tip. A delivery target substance may be inserted into the engraved groove, and thus may be stably implanted to the body while the delivery target substance is not damaged during the implantation. The engraved groove may have a diameter of 50 μm to 1 mm, and preferably, 100 μm to 500 μm. The engraved groove may have a depth of 50 μm to 2 mm, and preferably, 100 μm to 700 μm.

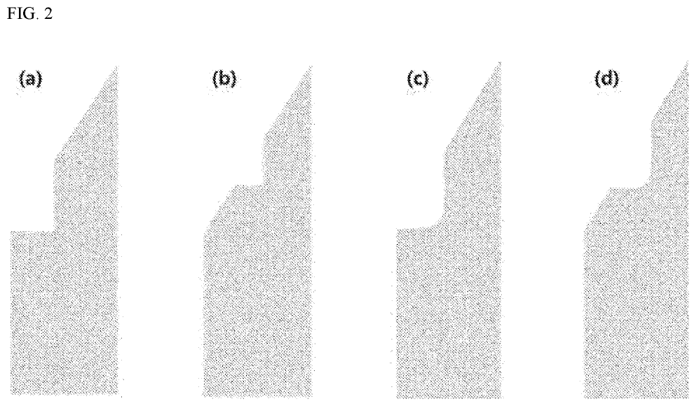

A position of the engraved groove may be adjusted such that the delivery target substance may be accurately inserted onto the surface of the bevel tip and at a desired location thereof In this regard, referring to FIG. 2, a shape and a position of the engraved groove will be described in more detail.

Referring to FIG. 2, as shown in (a) and (b), the engraved groove may have an L-shape in a vertical cross-section of the pin. As shown in (c) and (b), the groove may have an L-shape with a rounded corner.

Regarding the position of the engraved groove, the groove may be formed in all areas of the surface of the bevel tip, and the position of the engraved groove may be adjusted in order to accurately implant the delivery target substance to a target implantation position. Preferably, as shown in (c) and (d) of FIG. 2, in the vertical cross section of the pin, the groove may be formed in the surface of the bevel tip located closer to the shorter side among the pair of parallel sides extending in the vertical direction. In the implantable substance delivering carrier including the engraved groove formed in this shape, it is easier for the delivery target substance to be caught in and detached from the tissue, so that it may be more effective to implant the delivery target substance to an accurate location.

Referring back to FIG. 1, the delivery target substance may be an exosome, protein, peptide, spheroid cell body, hydrogel, or dried solid body, and may have a spherical or oval shape. The delivery target substance may have a form of a core-shell in which the delivery target substance is located in a core and a biodegradable polymer or hydrogel surrounding the substance is located in a shell. Due to the shell made of the biodegradable polymer or hydrogel, the delivery target substance may be delivered into the body and may have stability in the body. Evaporation of moisture in the delivery target substance may be prevented due to the shell.

The delivery target substance inserted into the engraved groove so as so as to protrude outwardly from the tip may be inserted into the groove so as so as to protrude outwardly beyond an imaginary line extending upwardly from the shorter side among the pair of parallel sides extending in the vertical direction in the vertical cross section of the pin. In this regard, when the delivery target substance is in the form of the core-shell in which the delivery target substance is located in a core and a biodegradable polymer or hydrogel surrounding the substance is located in a shell, the shell made of the biodegradable polymer or hydrogel may protrude outwardly beyond the imaginary line, or the core as the delivery target substance may protrude outwardly beyond the imaginary line.

In one embodiment, the implantable substance delivering carrier may further include a coating layer covering the delivery target substance and an entirety or a portion of the surface of the bevel tip. Even when the delivery target substance is inserted into the groove so as to protrude outwardly from the tip, the delivery target substance is prevented from being damaged or dropped due to the coating layer and thus may be stably transplanted into the tissue when the implantable substance delivering carrier is inserted into the tissue. Further, the delivery target substance may be easily caught on the tissue due to a jaw formed by the coating layer, thereby inducing stable desorption of the delivery target substance while reducing the impact applied to the substance.

A method for manufacturing an implantable substance delivering carrier according to the present disclosure includes: dripping a delivery target substance dispersed in a polymer solution into an engraved groove of a pin; and drying the solution.

The polymer solution may be a biopolymer solution, specifically, hyaluronic acid methacrylate.

In the step of positioning the delivery target substance in the engraved groove, the delivery target substance positioned in the engraved groove may be in a form in which the delivery target substance is positioned in a core and the polymer solution surrounding the core is located in a shell.

After the positioning step, the drying step may be performed. In the drying step, when the delivery target substance is located in the core and the polymer solution surrounding the same is located in the shell, and as the polymer solution dries, the delivery target substance may be naturally seated in the groove due to surface tension, and the shell surrounding the delivery target substance may be formed by crosslinking the polymer solution.

The drying may be dried using UV. However, the disclosure is not necessarily limited thereto. It is preferable to dry the solution to an extent such that when the implantable substance delivering carrier is inserted into a target tissue, the delivery target substance is not removed from the implantable substance delivering carrier.

In the implantable substance delivering carrier prepared using the manufacturing method, the coating layer covering the delivery target substance and an entirety or a portion of a surface of the bevel tip may be formed.

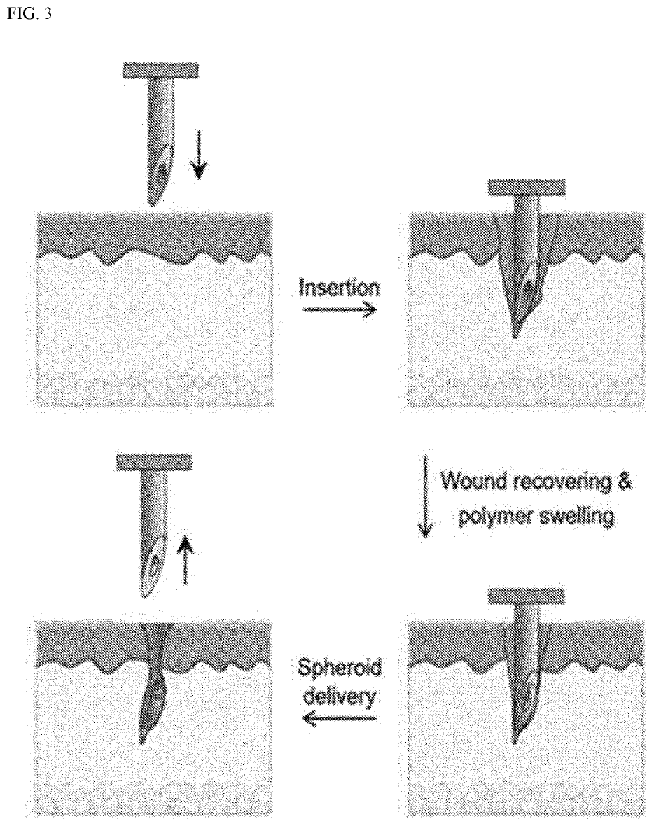

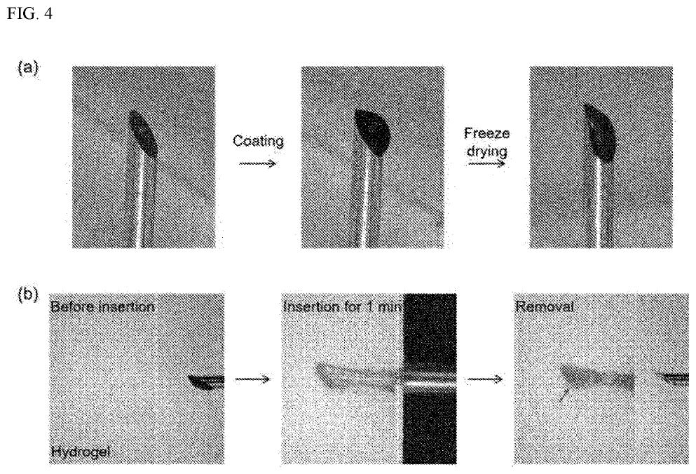

Hereinafter, with reference to FIG. 3 and FIG. 4, a process of implanting the delivery target substance using the implantable substance delivering carrier according to the present disclosure will be described in detail.

First, referring to FIG. 3, the implantable substance delivering carrier according to the present disclosure is inserted at a desired location of the tissue to which the delivery target substance is to be implanted. Then, the delivery target substance is implanted into the tissue while withdrawing the pin of the implantable substance delivering carrier out of the tissue. In this regard, the delivery target substance inserted into the engraved groove so as to protrude outwardly from the tip may be caught in the tissue and separated and detached from the pin of the implantable substance delivering carrier, and the separated and detached delivery target substance may be stably transplanted into the tissue.

In a conventional coated implantable substance delivering carrier (e.g., a needle, etc.), the coated substance is dissolved in the tissue such that the delivery target substance is implanted thereto. However, in accordance with the present disclosure, the delivery target substance inserted into the engraved groove so as to protrude outwardly from the tip of the implantable substance delivering carrier may be caught on the tissue and detached from the tissue, and thus the implantable substance may be stably and accurately implanted thereto.

In this regard, the delivery target substance caught on the tissue may be in the form of the core-shell composed of the core made of the delivery target substance and the shell made of a biodegradable polymer or hydrogel surrounding the delivery target substance.

In addition, when the implantable substance delivering carrier including the coating layer covering the delivery target substance and an entirety or a portion of the surface of the bevel tip is used, the coating layer is caught in the tissue and the delivery target substance together with the coating layer is detached from the tissue.

In this regard, referring to FIG. 4, the process of implanting the delivery target substance using the implantable substance delivering carrier according to the present disclosure will be described in more detail. First, in (a), the polymer solution containing the substance to be delivered (i.e., the delivery target substance) is dripped on the pin, coated, and freeze-dried such that the solidified delivery target substance is inserted into the groove of the implantable substance delivering carrier according to the present disclosure. It may be identified that the delivery target substance or a combination of the delivery target substance and the polymer solution is well solidified via the freeze-drying. Further, it may also be identified that the delivery target substance or the combination of the delivery target substance and the polymer solution is inserted into the engraved groove so as to protrude outwardly from the tip. Further, in (b), the process of implanting the delivery target substance using the implantable substance delivering carrier according to the present disclosure as prepared as in (a) may be identified. In this regard, it may be identified that when the carrier is inserted into the skin, the freeze-dried solid substance of the implantable substance delivering carrier is removed and detached from the pin while being caught on the tissue, and thus is stably implanted into the tissue.

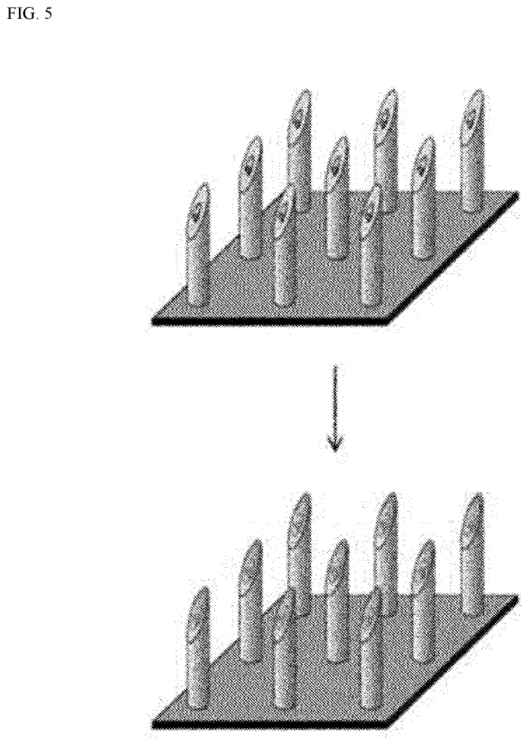

Further, referring to FIG. 5, in the implantable substance delivering carrier according to the present disclosure, the length of the pin and the position of the engraved groove may be adjusted to quantitatively implant single or multiple delivery target substances to the target locations once.

Hereinafter, an implantable substance delivering carrier according to the present disclosure and a method for manufacturing the same will be described in more detail based on specific present examples and comparative examples. However, the examples of the present disclosure are only some embodiments of the present disclosure, and the scope of the present disclosure is not limited to the following examples.

### EXAMPLE 1: Preparation of Carrier for Delivery of Implantable Substance

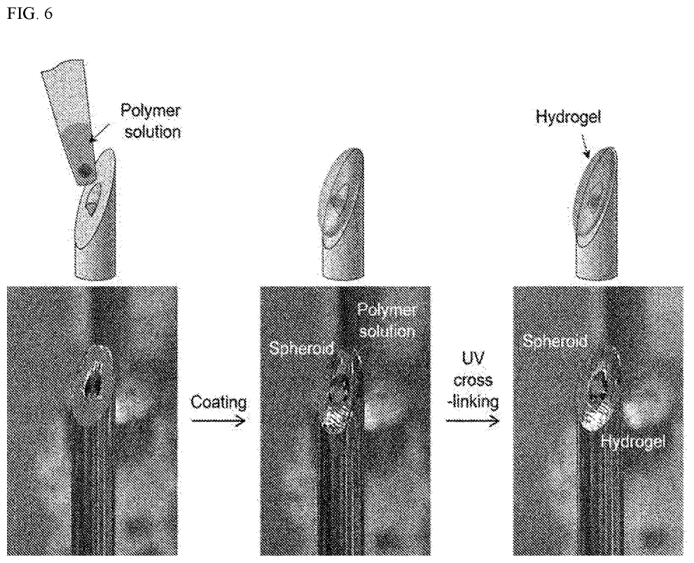

Referring to FIG. 6, the preparation of the implantable substance delivering carrier according to the present disclosure according to Example 1 of the present disclosure will be described.

Referring to FIG. 6, in order to prepare an implantable substance delivering carrier according to the present disclosure, the pin having the groove of 300 μm engraved in the surface of the bevel tip with a 55° angle was used. A spheroid was used as the delivery target substance, and the spheroid was prepared via three-dimensional culture of fibroblasts (NIH3T3). A solution was prepared by dispersing the spheroids in hyaluronic acid methacrylate (hereinafter referred to as HAMA). The solution was used to position the spheroids in the engraved groove, and then, was dried. In this way, the implantable substance delivering carrier according to Example 1 of the present disclosure was prepared.

It may be identified that the prepared carrier for delivering the implantable substance includes a HAMA coating layer covering the spheroid and an entirety or a portion of the surface of the bevel tip.

### Experimental Example 1: Evaluation of Cell Viability

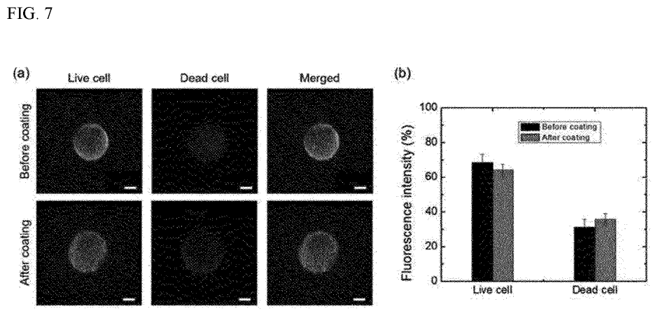

In the implantable substance delivering carrier according to the present disclosure, in order to evaluate the cell viability before and after the HAMA coating, the implantable substance delivering carrier was prepared through the same process as in Example 1 of the present disclosure, and after 10 minutes, the spheroid was collected again to identify live and dead cells. Live cells were stained with green fluorescence, and dead cells were stained with red fluorescence, and images were obtained using a microscope. The results are shown in FIG. 7.

Referring to FIG. 7, it may be identified from (a) that all the live cells, dead cells, and merged cells exhibited the same color before and after the coating. Further, it may be identified from (b) in FIG. 7 that the cell viability graphs of the live cells and dead cells have similar result values.

Thus, it may be identified that the delivery target substance according to the present disclosure is not damaged during the coating and the curing.

### Experimental Example 2: Evaluation of Transplantation Efficiency

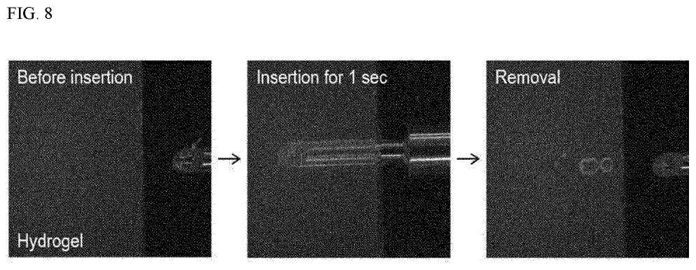

In the implantable substance delivering carrier according to the present disclosure, in order to evaluate the implantation efficiency, the implantable substance delivering carrier prepared through the same process as in Example 1 was inserted into a desired position of an agar gel based artificial skin having a strength similar to that of a real skin, and then the pin was pulled out. Then, it was checked whether the spheroid was stably implanted into the correct position. The results are shown in FIG. 8.

Referring to FIG. 8, it may be identified that the substance may be successfully implanted to the desired location using the implantable substance delivering carrier according to the present disclosure.

Although the above disclosure has been described with reference to a preferred embodiment of the present disclosure, those skilled in the art may variously modify and change the present disclosure within the scope not departing from the spirit and scope of the present disclosure described in the following claims.

1. An implantable substance delivering carrier comprising:
a pin including a bevel tip and a groove engraved in a surface of the bevel tip; and
a delivery target substance inserted into the engraved groove so as to protrude outwardly from the tip.

2. The implantable substance delivering carrier of claim 1, wherein the delivery target substance has a spherical or oval shape.

3. The implantable substance delivering carrier of claim 1, wherein the delivery target substance includes exosome, protein, peptide, spheroid cell body, hydrogel or dried solid body.

4. The implantable substance delivering carrier of claim 1, wherein the delivery target substance has a form of a core-shell in which the delivery target substance is located in the core and a biodegradable polymer or hydrogel surrounding the core is located in the shell.

5. The implantable substance delivering carrier of claim 1, wherein the implantable substance delivering carrier further includes a coating layer covering the delivery target substance and an entirety or a portion of the surface of the bevel tip.

6. The implantable substance delivering carrier of claim 1, wherein the delivery target substance is inserted into the grove so as to protrude outwardly beyond an imaginary line, wherein the line extends upwardly from a shorter side among a pair of parallel sides of the pin extending in a vertical direction in a vertical cross section of the pin.

7. The implantable substance delivering carrier of claim 1, wherein the engraved groove has an L-shape or an L-shape with a rounded corner in a vertical cross-section of the pin.

8. The implantable substance delivering carrier of claim 1, wherein the engraved groove is formed in the surface of the tip closer to a shorter side among a pair of parallel sides of the pin extending in a vertical direction in a vertical cross section of the pin.

9. The implantable substance delivering carrier of claim 1, wherein the surface face of the bevel tip is inclined at an angle of 45° to 80°.

10. The implantable substance delivering carrier of claim 1, wherein a length of the pin is in a range of 300 μm to 30 cm.

11. (canceled)

12. The implantable substance delivering carrier of claim 1, wherein a thickness of the pin is in a range of 200 μm to 3.4 mm.

13. The implantable substance delivering carrier of claim 1, wherein a diameter of the engraved groove is in a range of 50 μm to 1 mm.

14. (canceled)

15. The implantable substance delivering carrier of claim 1, wherein a depth of the engraved groove is in a range of 50 μm to 2 mm.

16. (canceled)

17. A method for manufacturing the implantable substance delivering carrier of claim 1, wherein the method comprises:
dripping the delivery target substance dispersed in a polymer solution into the engraved groove of the pin; and
drying the solution.

18. The method of claim 17, wherein the method further comprises forming a coating layer covering the delivery target substance and an entirety or a portion of the surface of the bevel tip.

19. The method of claim 17, wherein the polymer solution includes hyaluronic acid methacrylate.
